(19) United States
(12) Patent Application Publication
(10) Pub. No.: US 2021/0381889 A1
(43) Pub. Date: Dec. 9, 2021

(54) WAVELENGTH-SWEPT LIGHT SOURCE

(71) Applicant: OFS Fitel, LLC, Norcross, GA (US)

(72) Inventor: Philip G Westergaard, Frederiksberg C (DK)

(73) Assignee: OFS Fitel, LLC, Norcross, GA (US)

(21) Appl. No.: 17/289,146

(22) PCT Filed: Oct. 31, 2019
(86) PCT No.: PCT/US19/59061

§ 371 Date: Apr. 27, 2021

Related U.S. Application Data
(60) Provisional application No. 62/754,082, filed on Nov. 1, 2018.

(51) Int. Cl. G01J 3/02; G01J 3/10; G01B 9/02; H01S 3/067

(52) U.S. Cl. CPC G01J 3/0218; H01S 3/06754; G01B 9/02091; G01J 3/10

(57) ABSTRACT

A wavelength-swept optical source is based upon a combination of a coherent source of ultra-short optical pulses, doped fiber amplifier, and specialized dispersive optical medium to create time-stretched pulses. The pulses are broadened to have a spectral bandwidth that covers a wavelength range of interest for a particular wavelength sweeping application and are thereafter subjected to time-stretching within the dispersive optical medium so as to sufficiently separate in time a number of wavelength components within each pulse.

### CROSS-REFERENCE TO RELATED APPLICATIONS

This application claims the benefit of U.S. Provisional Application No. 62/754,082, filed Nov. 1, 2018, and herein incorporated by reference.

### TECHNICAL FIELD

The present invention relates to a wavelength-swept light source and, more particularly, to a source that is able to provide a wavelength-swept output over a broad spectral range at a relatively fast scan rate useful for imaging, sensing, and spectroscopy applications (e.g., a scan rate exceeding 2 MHz).

### BACKGROUND OF THE INVENTION

Beyond the use of optical systems for communications applications, the use of is laser-based arrangements in imaging, sensing, and spectroscopy applications have proved to be a valuable technique for capturing and analyzing data. In a variety of these systems, it is useful to have what is sometimes referred to as a “broadband” light source, and what is more properly characterized as a “wavelength-swept” light source, where a series of beams at a defined set of wavelengths is used to illuminate a given object. Since the object's response is typically a function of the wavelength of the illuminating light, the act of “sweeping” a set of different wavelengths across an object provides a characteristic wavelength-dependent response that may be able to, for example, sense the presence of a poisonous gas, recognize the presence of a slight deformation in a bridge span, or even assist in characterizing a tumor found in the human body.

In order to provide consistent and repeatable results, it is important for the laser source used to create a swept wavelength output to exhibit as high a coherence level as possible. Indeed, some applications may require a coherence length of at least 1 mm (“coherence length” being a span over which there is a well-defined phase relationship between the start and the end of the propagating light wave). A popular choice of a laser source for providing a swept-wavelength output with this level of coherence is a “Fourier Domain Mode-Locking” (FDML) laser. In an FMDL laser, the output wavelength is varied by tuning a variable bandpass filter disposed within the laser cavity. The tuning typically involves some type of actuation to adjust the filter's center wavelength (typically mechanical or, at times, thermal), thus limiting not only the scan speed (i.e., the time required sweep across a wavelength range from one end to the other), but also the “duty cycle” of the source, since the tunable filter needs to be re-set to the initial wavelength value before beginning the next sweep.

The need for any type of external actuator/filter mechanism to control a wavelength-swept light source thus inherently limits the sweep rate and/or bandwidth that may be achieved, especially since most attempts to improve their performance adds to the complexity, size and expense of the final product.

### SUMMARY OF THE INVENTION

The present invention relates to a wavelength-swept light source and, more particularly, to a fiber-based source that is able to provide a wavelength-swept output over a broad spectral range at a scan rate well in excess of prior art arrangements, without the need to perform any actuation-based tuning of the output wavelength.

In accordance with the principles of the present invention, a wavelength-swept light source is formed of a combination of a coherent pulsed laser source, a fiber-based optical amplifier, and a dispersive optical medium (in most cases embodied as a section of dispersive optical fiber). The parameters of these elements are coordinated so that the output from the dispersive optical medium consists of a series of “time-stretched” pulses, where selected wavelength components within a given stretched pulse exit the light source at measurably different (i.e., “distinct”) points in time. By mapping a set of wavelength components to specific arrival times via the dispersive Fourier transform (DFT) technique, instrumentation disposed at the output of the wavelength-swept light source will be able to correlate a time sequence with the defined wavelength components within each time-stretched pulse exiting the wavelength-swept light source.

Advantageously, the use of the “time-stretched” pulses to create a wavelength-swept light output eliminates the need to use a tunable bandpass filter to generate the wavelength sweep, allowing for a significant increase in the scan rate over prior art configurations. Also, since there is no need to manually “re-set” a tunable filter between cycles, the wavelength-swept light source of the present invention is able to utilize higher repetition rate input pulse sources than the prior art. Indeed, a repetition rate of 4.7 MHz has been used in the testing of exemplary fiber-based wavelength-swept light sources of the present invention.

The operating parameters of the various elements of the inventive wavelength-swept source are coordinated to provide an acceptable level of output power uniformity over across a bandwidth range of interest. For example, embodiments of the present invention are capable of achieving a variation in power spectral density (PSD) of less than 10 dB over a relatively wide spectral range by proper selection of operating parameters of the doped-fiber amplifier element.

In one or more embodiments, the coherent pulsed laser source may comprise a mode-locked fiber laser (e.g., a figure-8 fiber laser) to provide ultrashort (1 ps or less) “seed” pulses as the input to the amplifier element.

The dispersive optical medium may comprise a fiber, waveguide, bulk optic device, or any other medium suitable for supporting the propagation of an optical signal. In preferred embodiments, the dispersive optical medium is preferably configured to exhibit a total dispersion that provides a duty cycle close to unity. For the purposes of the present invention, the term “duty cycle” as used here is defined as the ratio of the time required to perform a complete wavelength sweep (t_{sweep}) to the complete cycle time interval (t_{cycle}).

An exemplary embodiment of the present invention may take the form of a wavelength-swept light source comprising a laser source of optical pulses (preferably ultra-short pulses), a doped-fiber optical amplifier, and a dispersive optical medium positioned at the output of the doped-fiber optical amplifier. The doped-fiber amplifier is responsive to both the optical pulses and a pump beam (of selected wavelength and power) to create spectrally-broadened output pulses having a minimal variation in power spectral density over a predetermined bandwidth within the spectrally-broadened bandwidth. The dispersive optical medium is configured to have an average pre-unit-length dispersion D_{avg} and predetermined length L_{DF} (defined as a total dispersion Dₜₒₜ of D_{avg}*L_{DF}), sufficient to “time-stretch” the amplified pulses from the doped-fiber optical amplifier such that different wavelength components within a pulse exit the dispersive optical medium at different points in time.

Another embodiment of the present invention relates to a method of generating a wavelength-swept optical output from a light source, the method comprising the steps of: providing a series of optical pulses at a predetermined repetition rate, applying the optical pulses as an input to a fiber-based optical amplifier, amplifying the optical pulses and broadening each pulse to span a predetermined spectral bandwidth, and then passing each pulse through a dispersive optical medium having a predetermined average chromatic dispersion D_{avg} and a predetermined length L_{DF} (creating a total dispersion Dₜₒₜ as defined above) for time stretching each spectrally broadened, amplified received at a dispersive medium. The transformed input optical pulses thus exit the dispersive optical medium as time-stretched pulses with different wavelength components of each pulse exiting the dispersive optical medium at different points in time, forming a wavelength-swept optical output.

Additionally, one or more embodiments of the present invention may take the form of a system comprising a short pulse seed input having a seed average power of a predetermined value and a repetition rate of a predetermined value, a pump laser diode generating a pump signal, a wavelength division multiplexer (“WDM”) combining the seed input and the pump signal, and a dispersive medium having a length L_{DF}, wherein the spectral width of the amplified optical source and the repetition rate of the short pulse seed input match a dispersion amount provided by the dispersive medium such that no wavelength component of a stretched pulse overlaps with a subsequent pulse.

Other and further aspects and embodiments of the present invention will become apparent during the course of the following discussion, and by reference to the associated drawings.

### BRIEF DESCRIPTION OF THE DRAWINGS

Referring now to the drawings, where like numerals represent like parts in several views:

FIG. 1 is a simplified block diagram of the various elements forming a wavelength-swept light source of the present invention;

FIG. 2 contains plots of time-stretched optical pulses, illustrating the relationship between the “sweep time” associated with the arrival of different wavelength components within a stretched optical pulse and the “cycle time” associated with the repetition rate of the coherent pulses used as the input to the wavelength-swept source;

FIG. 3 is a plot of an exemplary spectrum created by a wavelength-swept light source of the present invention, illustrating the power spectral density (PSD) as a function of wavelength (bottom scale), with arrival times of the light at a photodetector shown along the top scale (defining the “sweep time”);

FIG. 4 illustrates an exemplary embodiment of a fiber-based wavelength-swept light source formed in accordance with the principles of the present invention; and

FIG. 5 illustrates an alternative embodiment of a fiber-based wavelength-swept light source formed in accordance with the principles of the present invention, in this case including a delivery fiber disposed between the doped-fiber amplifier and the dispersive fiber output element.

### DETAILED DISCUSSION

Wavelength-swept sources for applications such as optical coherence tomography (OCT) have typically comprised tunable lasers. These lasers are known to exhibit a high spectral brightness and require only a relatively simple optical design to create the required tuning across the available wavelength range. As mentioned above, conventional arrangements employ a type of wavelength tuning that involves some mechanical actuation (e.g., of a movable bandpass filtering element), thus limiting not only the maximum scan speed, but also the duty cycle of the device.

Optical-based “time stretching” is an all-optical technique that does not require any type of mechanical control of tuning. Rather, an optical element (bulk device, waveguide, fiber, etc.) is used to spread out an incoming pulse as a function of time. That is, the dispersion characteristic of the optical element serves to control the arrival times of the various wavelength components within the optical pulse. This so-called time stretching technique (also referred to at times hereafter as a “dispersive Fourier transform” (DFT) technique) results in the ability to provide wavelength-to-time mapping, thus creating an effective sweep of wavelengths over time. In accordance with the principles of the present invention, the DFT technique may be utilized in conjunction with the output from the inventive wavelength-swept source to provide wavelength sweeping over a relatively large spectral range (in excess of 100 nm), with a relative uniform power distribution across the individual wavelength components (e.g., deviation less than 10 dB), without experiencing the limitations in scan rate that are associated with “moving part” prior art arrangements.

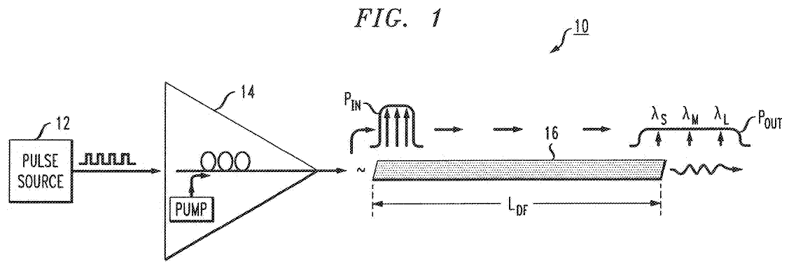

FIG. 1 is a block diagram illustrating the various elements used to form a wavelength-swept light source 10 in accordance with the principles of the present invention. While the elements are shown in this diagram as discrete, separate components, it is to be understood that each element is preferably formed of sections of optical fiber configured to exhibit characteristics selected to generate a wavelength-swept output of the desired spectral bandwidth and scan rate suitable for a particular application.

As shown in FIG. 1, wavelength-swept light source 10 comprises a laser pulse source 12 that is used to supply a train of coherent optical pulses at a defined repetition rate (preferably, “ultra-short” pulses with a pulse duration of 1 ps or less). Both the pulse duration and the repetition rate are parameters that may be specifically determined and designed to provide a wavelength-swept output that meets the requirements of a particular application. The power level of these pulses, as well as their coherence, are other factors important in the generation of a wavelength-swept output. At various times, these pulses will be referred to as “seed” pulses, a term well-known in the art for defining a system input used to trigger a sequence of events that creates a desired output.

A “highly coherent” laser is preferred for use as pulse source 12, since there are applications for a wavelength-swept light source where the coherence length should be as long as possible. For example, optical coherence tomography (OCT) imaging techniques require a coherence length of at least some minimal value (e.g., 1 mm or so). By “coherence” it is meant that there is a predictable phase relationship between one or more consecutive pulses.

The pulse train output from source 12 is thereafter applied as an input to a doped-fiber optical amplifier 14, which is used to inject a controllable amount of gain and spectral broadening to each pulse, creating a spectral bandwidth Δv that is used to define the upper and lower limits of the wavelength sweep range provided by the inventive wavelength-swept light source. Additionally, it is an important aspect that doped-fiber optical amplifier 14 provide a relatively smooth power distribution across the spectral bandwidth Δv. As will be discussed in detail below, while it is a goal to provide as wide a bandwidth as possible, this comes at the cost of increasing the pump power of amplifier 14 to the point where unwanted nonlinear effects degrade the uniformity of the power distribution. One exemplary embodiment described below configures both the pump power and absorption property of the gain fiber to achieve a spectral bandwidth of about 130 nm, with less than 10 dB variation in power spectral density (PSD) across this bandwidth.

Continuing with the description of the components of FIG. 1, the relatively high power, spectrally broadened output pulses from doped-fiber amplifier 14 are thereafter coupled into a dispersive optical element 16. As will be discussed in detail below, the chromatic dispersion (D) of this element is a key factor in configuring an element that is able to sufficiently separate (in time) the wavelength components within a pulse so that specific wavelengths arrive at the output of source 10 at well-separated points in time (“well-separated”, “distinct”, and the like referred to herein as describing a time interval that allows for an associated photodetecting device to accurately measure the optical power in each separate wavelength component). Various types of dispersive medium may be used to form element 16, including bulk optic nonlinear components, waveguide-based components, and fiber-based components.

The pulse stretching and “wavelength-to-time” mapping aspects of the present invention are illustrated in association with dispersive element 16 in FIG. 1, which shows an input pulse P_{IN} having a relatively high power level (upon its exit from amplifier 14). Thereafter, as the pulse propagates along dispersive element 16, the specific chromatic dispersion property of this element functions to modify the propagation velocity of different wavelength components within the pulse, resulting in the “time-stretched” appearance of the same pulse at the output of wavelength-swept light source 10 (shown as P_{OUT} in FIG. 1).

In most cases, an optical element's chromatic dispersion is such that light at longer wavelengths travels faster than light at shorter wavelengths (variously referred to as “normal” or “negative” dispersion, the value is measured in terms of ps/nm-km). However, it is also possible to design a dispersive optical medium to exhibit positive dispersion (also referred to at times as “anomalous dispersion”), where light at shorter wavelengths travels faster than light at longer wavelengths. While in general it is possible to utilize either type of dispersive element in the wavelength-swept light source of the present invention, the use of normal/negative dispersion element is typically preferred and may be formed to exhibit an acceptably uniform dispersion across the spectral bandwidth Δv. Reference is made to U.S. patent application Ser. No. 15/970,990, assigned to the assignee of this application and thus herein incorporated by reference, which describes details related to a high “figure of merit” (FOM) optical fiber that is suitable for use as dispersive optical element 16.

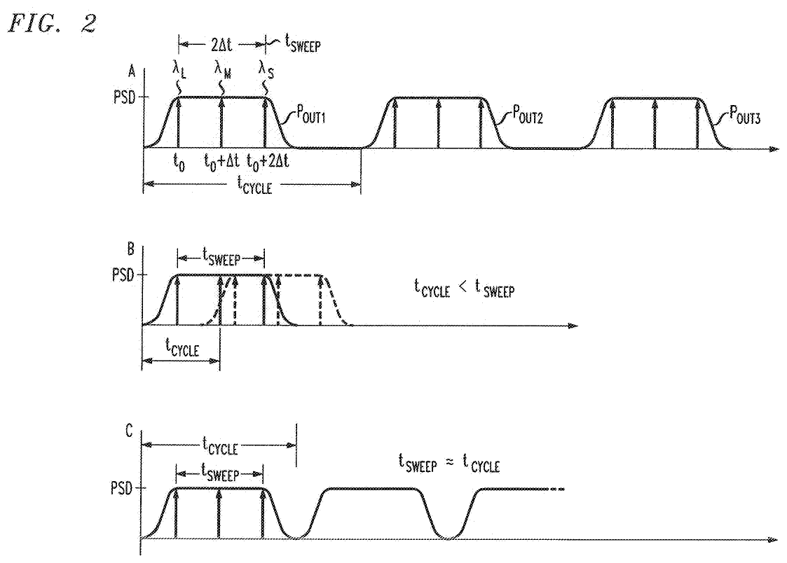

Before describing specific embodiments of the present invention, it is useful to consider the relationship between the inverse of the repetition rate of the seed pulses produced by pulse source 12 (also referred to as the “cycle time”) and the “wavelength sweep duration”. For the purposes of the present invention, the ratio of these two time intervals is defined as the “duty cycle” of wavelength-swept light source 10. The diagrams of FIG. 2 illustrate this aspect of the present invention.

Plot A of FIG. 2 is a plot of exemplary time-stretched output pulses P_{OUT} as exiting dispersive optical element 16. The pulses (which are shown in idealized form for illustrative purposes) are plotted to show their power spectral density (PSD) as a function of time. To simplify the following discussion, it is presumed that only a set of three distinct wavelength components are used to form the “sweep” (i.e., the light output from dispersive optical element 16 is defined as including a set of three spaced-apart wavelength components, denoted here as: λ_{L}, λ_{M}, and λ_{S}). As mentioned above, the DFT technique may be used to perform the function of mapping these time-based measurements to actual wavelength values. The mapping may then be used in conjunction with wavelength-swept light source 10 to allow for an operator of the wavelength-swept source to calibrate the on-going arrival times of a sequence of time-stretched output pulses from wavelength-swept light source 10 to a set of known wavelength values. A set of three time-stretched output pulses are shown in plot A as P_{OUT1}, P_{OUT2}, and P_{OUT3}.

The wavelength sweep time duration is shown in plot A as time interval t_{sweep} (i.e., the elapsed time 2Δt). The interval t_{sweep} is a function of the dispersion introduced to the pulse by dispersive optical element 16; that is, the time stretch now introduced between the arrival of wavelength component λ_{L} at time=t₀ and the arrival of wavelength component λ_{S} at time=(t₀+2Δt). The “cycle time” t_{cycle} is shown in plot A as the elapsed time period between the rise time of P_{OUT1} and the rise time of P_{OUT2}. The cycle time may also be defined by its inverse, the “repetition rate” (fᵣₑₚ) of the seed pulses. In embodiments described below, embodiments of the present invention are able to function at a repetition rate of 4.7 MHz (a cycle time of about 200 ns), while maintaining a relatively smooth PSD profile over a spectral bandwidth Δv of at least 130 nm.

The duty cycle associated with the exemplary output time-stretched pulse train shown in plot A has a value on the order of about one-half, since t_{sweep} is shown as extending across about half of the total cycle time. While acceptable, inasmuch as the fiber-based source of the present invention does not need to be “re-set” to an initial state to begin each subsequent sweep, it is clear that a longer sweep time may be used, allowing for additional wavelength components to be used within the sweep, or providing a higher-resolution output power measurement of the individual wavelength components, or both.

However, as mentioned above, there is a need to maintain the duty cycle of the inventive wavelength-swept source to a value less than unity. Plot B of FIG. 2 illustrates a situation to be avoided, where the duty cycle has increased to a value greater than one (i.e., where t_{sweep} is greater than t_{cycle}). As shown, this may lead to an out-of-sequence arrival of wavelength components at the output of wavelength-swept source 10 such that the trailing edge of one pulse overlaps with the rising edge of a subsequent pulse. Such an overlap between stretched pulses could be attributed to a repetition rate of the seed pulses that was too fast, or a total dispersion of dispersive optical element 16 that was too large.

Indeed, a preferred embodiment of the present invention is configured to provide a high duty cycle approaching unity (i.e., t_{sweep}≈t_{cycle}). This is possible since there is no need to re-set mechanical filter components before initiating a new sweep, so once the shortest wavelength of a first pulse has exited source 10, it is ready to transmit the longest wavelength component of the next pulse. Therefore, in accordance with the principles of the present invention, a wavelength-swept light source is provided that may utilize a scan rate that is essentially the same as (but not exceeding) the repetition rate of the original seed pulses.

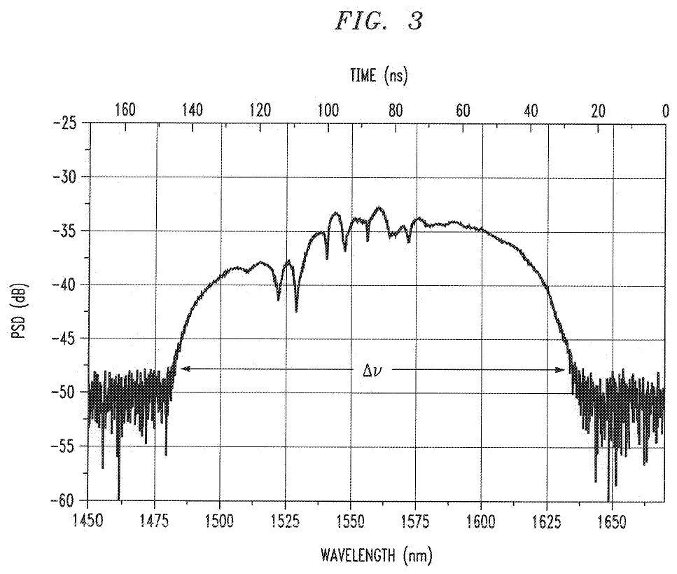

FIG. 3 is a power spectral density plot for an exemplary wavelength-swept spectrum as measured by a photodetector and correlated to specific wavelength values by the DFT technique. Consistent with conventional illustrations, the spectrum is plotted from “short” to “long” wavelength values, showing power spectral density (PSD) as a function of wavelength (measured in nm). A time scale is shown across the top of the plot, where the “arrival time” of the individual wavelength components reads from right to left (i.e., the higher-wavelength components arrive prior to the shorter-wavelength components). Here, a variation of less than 10 dB is maintained in the PSD over a spectral bandwidth Δv of about 130 nm, more than sufficient to provide a large number of separate wavelength components at essentially the same power level. The elapsed time for this bandwidth to be processed by detector 18 is shown to be about 100 ns.

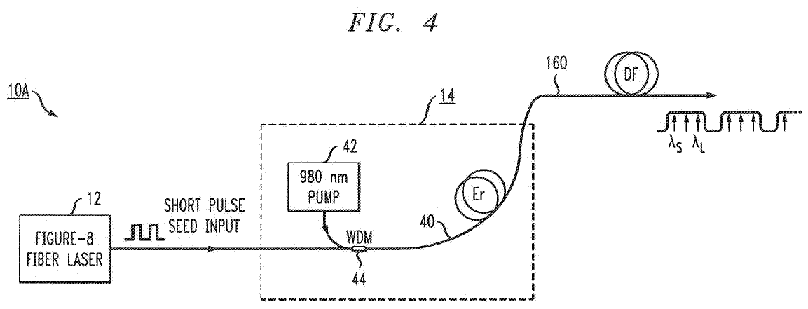

FIG. 4 illustrates in slightly more detail an exemplary wavelength swept light source 10A, based upon the principles discussed above in association with FIGS. 1-3 and defining several of the parameters that may be configured to obtain a wavelength-swept output of a defined spectral width and scan rate as required for a given application. In particular, there are parameters of all three components (i.e., pulse source 12, doped-fiber amplifier 14 and dispersive optical element 16) that may be particularly selected, designed, or adjusted as need be to meet the requirements of different applications.

Regarding specific attributes of pulse source 12, the configuration shown in this embodiment comprises a fiber-based laser that is capable of generating coherent ultra-fast seed pulses with an average power on the order of 300 μW, a pulse duration of around 250 fs and a repetition rate of 4.7 MHz (which translates to a cycle time on the order of 200 ns). A mode-locked “figure-8” laser, such as described in U.S. patent application Ser. No. 16/200,810 and assigned to the assignee of this application, is considered to be exemplary of a low-noise coherent laser source suitable for this purpose.

In the particular embodiment shown in FIG. 4, doped-fiber amplifier 14 is shown as comprising a section of erbium-doped gain fiber 40 and a pump source 42 for providing amplifying light at a wavelength of about 980 nm. A wavelength division multiplexer (WDM) 44 is included and used direct both the seed pulses from pulsed laser source 12 and the pump light from pump source 42 into Er-doped gain fiber 40.

In accordance with the principles of the present invention, doped-fiber amplifier 14 is configured to provide spectral broadening of the seed pulse, while providing an essentially uniform gain distribution over the created spectral bandwidth Δv. These characteristics are achieved in this embodiment by controlling the output power of pump source 42, in combination with the pump power absorption parameter of gain fiber 40. In particular, it is known that the spectral broadening may be related to the pump power level in certain cases, where as the pump power increases, the increased optical interactions along the gain fiber tend to increase the wavelength range of the output (i.e., “spectral broadening”). While a broader spectral range means that a larger number of individual wavelength components may be identified and used in the wavelength-swept output from source 10A, the increase in pump power necessary to achieve this may also result in amplifying unwanted noise components contained within the propagating wave or being created in the amplification process itself.

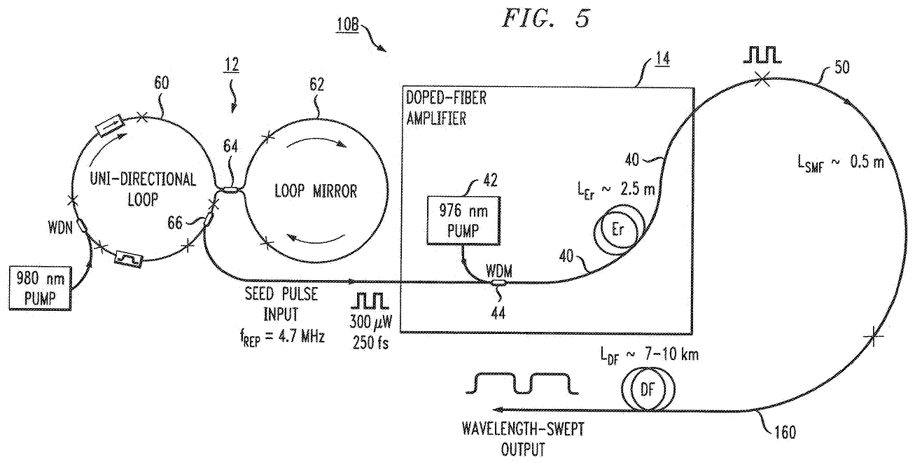

Thus, an important aspect of the present invention relates to determining an acceptable amount of gain over a specific spectral bandwidth Δv that is useful for a given application, without also amplifying noise components outside of this range. Indeed, there is an upper limit to the amount of gain that should be provided by doped-fiber amplifier 14, where too much gain has been found to induce detrimental non-linear effects such as self-phase modulation (SPM), cross-phase modulation (XPM), Raman scattering, and the like (generally referred to as “noise”). An “acceptable amount” of gain is thus associated with ensuring that doped-fiber amplifier 14 operates in a “low noise” regime. Specific ranges of acceptable values are discussed below in association with the embodiment of FIG. 5.

Continuing with the description of light source 10A as shown in FIG. 4, the amplified, spectrally-broadened pulses created by amplifier element 14 are thereafter coupled into dispersive optical element 16, which in this case comprises a section of dispersive fiber 160, shown as having a defined length L_{DF}. As discussed above, each pulse passing through dispersive fiber 160 is “stretched” in time, so that different wavelength components within the pulse arrive at the output of light source 10A at measurably-different points in time.

In some embodiments of the present invention, the length L_{DF} of dispersive fiber 160 may be optimized to provide a duty cycle close to unity for the reasons discussed above in association with FIG. 2. Indeed, a duty cycle approaching unity has been found to provide improved spectral resolution for a given detection bandwidth (this bandwidth typically defined as the combination of photodetector response time and digitizer bandwidth). An approximate value of the optimized length for dispersive fiber 160 (L_{DF,opt}) may be obtained from the following formula:

$${L}_{DF , opt} ≃ \frac{{t}_{cycle}}{{D}_{avg} \cdot Δ v} = {( {f}_{rep} \cdot {D}_{avg} \cdot Δ v )}^{- 1} ,$$

where D_{avg} is the average chromatic dispersion value of dispersive fiber 160 over the bandwidth in question, and the other terms in the relation are as defined above.

Another embodiment of the present invention, referred to as wavelength-swept light source 10B, is shown in FIG. 5. In this particular embodiment, an additional section of optical fiber is included within the wavelength-swept light source. In particular, a section of optical fiber 50 is shown as disposed between the output of amplifier 14 and the input to dispersive fiber 16. Also referred to at times as a “delivery” fiber, optical fiber 50 may be included in applications where doped-fiber amplifier 14 cannot be positioned in relative close proximity to dispersive element 16, or if additional spectral broadening is desired before introducing the pulses to the dispersive medium. Moreover, it is contemplated that the inclusion of an additional section of standard single mode fiber between gain fiber 40 and dispersive fiber 160 allows for a pair of fusion-spliced connections (as marked by the X's in FIG. 5) to be used so as to maintain efficient coupling with little loss of power between the core regions of Er-doped gain fiber 40 and dispersive fiber 160. In an exemplary embodiment, optical fiber 50 may comprise a section of single mode optical fiber that is fused to end terminations of both Er-doped gain fiber 40 and dispersive fiber 160.

For the particular embodiment shown in FIG. 5, laser pulse source 12 is specifically illustrated as a figure-8 fiber-based laser (such as disclosed in U.S. patent application Ser. No. 16/200,810, referenced above), which includes a uni-directional fiber loop 60 and a bi-directional loop “mirror” 62, with an optical coupler 64 providing signal coupling between the two loops. An output coupler 66 is used to direct a portion of signal circulating around uni-directional fiber loop 60 (comprising mode-locked optical pulses) along an output path and into doped-fiber amplifier element 14.

Here, doped-fiber amplifier element 14 is shown as using a section of Er-doped fiber 40 that has a nominal absorption (of the propagating pump wave) on the order of about 27 dB/m. Pump source 42 is shown as providing a pump beam at a wavelength of 976 nm, and is set to operate in this case at a pump power of 250 mW. For this particular combination of amplifier parameters, when considered with the use of seed pulses having an input pulse energy of about 60 pJ (i.e., a power of 300 μW at a 4.7 MHz repetition rate), using an Er-doped gain fiber 40 of length L_{Er} on the order of about 2.5 m has been found to provide a relatively uniform power spectral density (PSD) over the spectral band of interest. In particular, for this set of parameters, output pulses from doped-fiber amplifier 14 have been found to exhibit a pulse energy of about 2-4 nJ (corresponding to an output power in the range of about 10-20 mW at the 4.7 MHz repetition rate), with a PSD of less than 10 dB over a spectral range of more than 130 nm.

It is to be understood that the specific values described above for the design of pulse source 12 and doped-fiber amplifier 14 are merely illustrative of values that coordinate in a manner that is useful in generating a wavelength-swept output in combination with an exemplary configuration of dispersive fiber 160, as will now be discussed with continuing reference to FIG. 5.

As mentioned above, a section of (single mode) delivery fiber 50 is included in fiber-based wavelength-swept light source 10B as shown in FIG. 5, with these high-power output pulses from doped-fiber amplifier 14 coupled into delivery fiber 50 so as to pass through and then be coupled into dispersive fiber 160. In the particular embodiment as illustrated in FIG. 5, dispersive fiber 160 is formed to exhibit an average chromatic dispersion value (D_{avg}) on the order of about −75 ps/nm/km. With this characteristic, a dispersive fiber of length L_{DF} of 7 km was found to provide a wavelength-swept output of the form shown in FIG. 3, having a spectral bandwidth Δv of about 130 nm and a variation in PSD of less than 10 dB over this bandwidth. As noted above, an important factor in configuring dispersive optical fiber 16 is to provide a controlled amount of dispersion across the complete spectral bandwidth. An exemplary dispersive fiber suitable for this purpose, referred to as a “high figure-of-merit” optical fiber, exhibits a relatively linear dispersion characteristic. U.S. patent application Ser. No. 15/970,990, entitled “Optical Fiber with Specialized Figure-of-Merit and Applications Therefore” and assigned to the assignee of this application includes a description of a particular type of dispersive fiber that is acceptable for use in a fiber-based wavelength-swept light source formed in accordance with the principles of the present invention.

A swept wavelength light source can therefore be constructed by combining a pulsed laser source with a suitable amount of dispersion for time stretching. The dispersion is preferably well-matched to the bandwidth and repetition rate of the input light source such that no wavelength component of a stretched pulse overlaps with the subsequent pulse. It is generally desirable to have as much output power and as wide a wavelength range as possible while maintaining a smooth distribution of power over the available spectral range, as well as a low level of power fluctuations from one pulse to the next.

The foregoing description of the invention is presented for purposes of illustration and description and is not intended to be exhaustive or to limit the invention to the precise form disclosed. Many modifications and variations are possible in light of the above teaching. The embodiments were selected and described to best explain the principles of the invention and its practical application to thereby enable others skilled in the art to best use the invention in various embodiments and with various modifications suited to the particular use contemplated.

What is claimed is:

1. A method of generating a wavelength-swept optical output from a light source, comprising:
providing a series of optical input pulses at a predetermined repetition rate;
amplifying and broadening each optical pulse of the series of optical input pulses within a fiber-based optical amplifier such that each amplified output pulse spans across a predetermined spectral bandwidth; and
passing each amplified and spectrally broadened output pulse through a dispersive optical medium having a predetermined average chromatic per-unit-length-dispersion D_{avg} and a predetermined length L_{DF}, providing a total dispersion Dₜₒₜ of (D_{avg}*L_{DF}), the dispersive optical medium performing time stretching on each propagating pulse by an amount sufficient to have different wavelength components of each time-stretched pulse exit the dispersive optical medium at spaced-apart time intervals, forming the wavelength-swept optical output.

2. The method as defined in claim 1 wherein the predetermined repetition rate and the predetermined spectral bandwidth are selected to correspond to the predetermined total chromatic dispersion Dₜₒₜ such that time-stretched pulses exiting the dispersive optical medium do not overlap in time.

3. The method as defined in claim 1 wherein the optical input pulses have a duration of less than 1 ps.

4. The method as defined in claim 3 wherein the optical input pulses having a duration of less than 300 fs.

5. The method as defined in claim 1 wherein the predetermined repetition rate of the optical input pulses is no less than 2 MHz.

6. The method as defined in claim 5 wherein the predetermined repetition rate of the optical input pulses is about 4.7 MHz.

7. The method as defined in claim 1 wherein the optical input pulses exhibit a coherence length of at least 1 mm.

8. The method as defined in claim 1 wherein the optical pulses exhibit an output pulse energy of about 60 pJ.

9. The method as defined in claim 1, wherein the wavelength-swept optical output exhibits a scan rate of at least 2 MHz.

10. The method as defined in claim 1 wherein the length of the dispersive optical medium is selected to essentially equal the inverse of the product of the pulse repetition frequency, average chromatic dispersion D_{avg} and spectral bandwidth.

11. A wavelength-swept light source, comprising:
a laser source of optical input pulses provided at a predetermined repetition rate;
a doped-fiber amplifier responsive to both the optical input pulses and a pump beam of selected wavelength and power, the doped-fiber amplifier creating spectrally-broadened output pulses having a minimal variation in power spectral density over a predetermined bandwidth within the spectrally-broadened region; and
a dispersive optical medium having an average chromatic per-length-dispersion D_{avg} and predetermined length L_{DF}, providing a total dispersion Dₜₒₜ defined as (D_{avg}*L_{DF}), the dispersive optical medium disposed to receive the amplified output pulses from the doped-fiber amplifier, wherein the output pulses from the doped-fiber amplifier are sufficiently time stretched at an exit of the dispersive optical medium such that a time interval from a first wavelength component to a last wavelength component of the time-stretched pulse is optimized with respect to the inverse of the predetermined repetition rate of the coherent optical input pulses.

12. The wavelength-swept light source as defined in claim 11
wherein the length (L_{DF}) of the dispersive optical medium is estimated as:
$${L}_{DF} ≃ \frac{{t}_{cycle}}{{D}_{avg} \cdot Δ v} = {( {f}_{rep} \cdot {D}_{avg} \cdot Δ v )}^{- 1} ,$$
wherein t_{cycle} is a temporal pulse interval, fᵣₑₚ is the pulse repetition rate, D_{avg} is an average dispersion of the dispersive fiber over the bandwidth value, and Δv is the spectral bandwidth of the light source.

13. The wavelength-swept light source as defined in claim 11 wherein the doped-fiber amplifier comprises an erbium-doped fiber amplifier, utilizing a pump source providing a beam at a nominal wavelength of about 980 nm, with a pump power of at least 200 mW.

14. The wavelength-swept light source as defined in claim 11 wherein the dispersive optical medium comprises a section of dispersive optical fiber.

15. The wavelength-swept light source as defined in claim 14 wherein the section of dispersive optical fiber exhibits an average absolute chromatic dispersion |D_{avg}| of at least 75 ps/nm-km, and exhibits a length L_{DF} in the range of about 7 km to about 10 km.

16. The wavelength-swept light source as defined in claim 14, further comprising a delivery optical fiber positioned between the doped-fiber amplifier and the dispersive optical fiber.

17. The wavelength-swept light source as defined in claim 16 wherein the delivery optical fiber comprises a single mode optical fiber.

18. The wavelength-swept light source as defined in claim 16 wherein the deliver optical fiber is fusion spliced in place between the doped-fiber amplifier and the dispersive optical fiber.

19. A system, comprising:
a short pulse seed optical input having a seed average power of a predetermined value and a repetition rate of a predetermined value;
a pump laser diode generating a pump signal;
a wavelength division multiplexer (“WDM”) combining the seed input and the pump signal; and
a dispersive medium having a length L_{DF}, wherein the spectral width of the amplified optical source and the repetition rate of the short pulse seed input match a dispersion amount provided by the dispersive medium such that no wavelength component of a stretched pulse overlaps with a subsequent pulse.

20. The system as defined in claim 19, wherein the system further comprises:
a doped-fiber amplifier for amplifying the short pulse seed input multiplexed with the pump signal and having a length L_{Er}.

21. The system as defined in claim 20, wherein the system further comprises:
a delivery optical fiber having a length L_{SMF} to transition between the fiber amplifier and the dispersive medium.

22. The system as defined in claim 19, wherein the short pulse seed input has a pulse energy of about 60 pJ, exhibited as an average power of approximately 300 μW for a repetition rate of approximately 4.7 MHz.

23. The system as defined in claim 18, wherein the pump laser diode generates the pump signal at approximately 200-300 mW at 970-980 nm.

24. The system as defined in claim 20, wherein the doped-fiber amplifier includes a section of Er-doped gain fiber of length L_{Er}≈2.3 m and absorbs approximately 27 dB/m of the pump signal to generate a pulse energy of approximately 2-4 nJ.

25. The system as defined in claim 19, wherein the system is implemented as a wavelength-swept light source within an optical coherence tomography (“OCT”) imaging system.
